(19) United States
(12) Patent Application Publication
(10) Pub. No.: US 2012/0130278 A1
(43) Pub. Date: May 24, 2012

(54) DEVICE FOR EXTRACTING BLOOD FROM THE PLACENTA AND/OR UMBILICAL CORD

(76) Inventor: Angel Gabriel Alvarez Ramos, Gijon (ES)

(21) Appl. No.: 13/386,927

(22) PCT Filed: Jul. 14, 2010
(86) PCT No.: PCT/EP2010/060131

§ 371 Date: Jan. 25, 2012

(30) Foreign Application Priority Data
Jul. 30, 2009 (ES) 200930536

(51) Int. Cl. A61B 5/00

(52) U.S. Cl. 600/583

(57) ABSTRACT

A device is provided to extract and collect blood from the placenta and/or umbilical cord, through a system that combines collecting the blood as it falls by the effect of gravity, together with a suction system that facilitates the fall. The device allows a greater amount of blood to be extracted than that obtained using conventional gravity methods, without entailing any risk to the mother's health, thereby allowing a controlled suction that prevents premature expulsion of the placenta.

### BACKGROUND OF THE INVENTION

1. Field of the Invention

The present invention relates to a device designed to simultaneously extract and collect blood from the placenta and/or umbilical cord, and more particularly, to a device based on collecting said blood while it falls through the effect of gravity, in combination with a suction system to facilitate its extraction.

2. Description of the Related Art

In recent years, the discovery of the existence of high stem cell content in the umbilical cord and placenta of pregnant women, has favoured the development of various systems for extracting and collecting the blood contained in said organs, with a view to storing it for subsequent use, or for scientific purposes, or for use, in case of need, for transplantation to patients suffering from various congenital or acquired diseases, such as immune deficiencies, cancerous diseases or metabolic disorders related to deposits, among others.

For transplants of blood from the umbilical cord and placenta to be effective, providing the therapeutic level required by the treated pathologies, it is of crucial importance to have a sufficient volume of stem cell-rich blood, requiring, at least, approximately 1.5×107 of precursor cells per Kg of the patient's weight for a successful implantation.

Currently, the majority of systems aimed at extracting blood from the umbilical cord and placenta, are based on mechanisms that pierce the umbilical cord, immediately after delivery of the baby, in the minutes prior to the expulsion of the placenta. Blood thereby extracted, falls through gravity, into a sack that collects the blood, said sack being held below the height of the woman who has just given birth, in such a way as to encourage the drop of blood. Normally, the sack contains anticoagulants and is kept in motion during the collection process, favouring the blood's preservation in optimum conditions.

By means of the foregoing method, the average volume of blood collected from each labour is estimated at approximately 90 ml. Although, in many cases, this volume is sufficient to obtain a stem cell content that a posteriori may be therapeutically effective, it also happens in a non-negligible number of patients that said volume does not manage to provide the required content in stem cells, due to insufficient cellularity.

Currently, the state of the art contemplates various systems designed to increase the amount of placental and umbilical cord blood extracted and collected, fundamentally using pressurised systems that allow said blood to be pumped, increasing thereby the collected volume and reducing the extraction time. Some examples of this type of system are the devices described in patents WO2005/041772, U.S. Pat. No. 5,097,842, U.S. Pat. No. 5,059,168 and U.S. Pat. No. 5,575,795. Said devices comprise one or more needles to pierce the umbilical cord and/or placenta, blood collection means, such as reservoirs, sacks, or vacuum containers, and suction or drainage means, such as vacuum pumps, peristaltic pumps, or syringes. In all of the references mentioned above, the amount of blood collected is increased by replacing the usual method, based on puncture and subsequent fall by gravity, with a method based exclusively on the negative pressure exerted by the suction or drainage means.

Although the aforesaid devices are capable of achieving the purposes for which they are designed, producing an increase in the amount of blood collected, they share a common problem related to their designed based on a suction method. As a result of continuously applying said suction, there is a possibility of precipitating the appearance of complications such as the sudden appearance of retroplacentary hematoma and, consequently, premature expulsion of the placenta before the necessary time has passed for said expulsion to occur naturally and for the maternal haemostasis factors that follow birth minimising the haemorrhage. This expulsion, which may be precipitated by an uncontrolled constant suction of blood, represents an obvious risk to the mother's health.

Therefore, it is necessary to develop devices that make it possible to increase the volume of blood collected from the placenta and umbilical cord, in such a way that the content of extracted stem cells is sufficient to fulfil the required therapeutic purposes, and that, at the same time, are safe for the mother in the phase immediately after giving birth, guaranteeing the natural expulsion of the placenta at no health risk whatsoever.

### SUMMARY OF THE INVENTION

The present invention is aimed at satisfying this need, by means of an extraction device that combines the usual system of blood falling by gravity, with the controlled assistance provided by a suction system.

Another advantage of the present invention is that, through a design that combines extraction by gravity and extraction by suction, it is possible to extract the blood contained in both the umbilical cord as well as the placenta by means of piercing only the umbilical cord. Not having to pierce the placenta is particularly relevant, since said puncture normally entails a risk of bacterial contamination avoided with the device described herein. Additionally, the possibility of extracting blood from the placenta by piercing the umbilical cord, makes it possible to extract blood immediately after the baby's delivery, without having to wait for the placenta to be expelled, which normally occurs within 5 and 30 minutes after giving birth, representing an optimisation in working times of the operating theatre, with the ensuing cost savings.

One object of the present invention is a device for extracting blood from the placenta and umbilical cord which simultaneously combines collecting blood as it falls due to the effect of gravity, with a suction system that facilitates said fall.

One object of the present invention is a device for extracting blood from the placenta and umbilical cord which comprises, at least, one blood extraction means consisting of, at least, one needle, catheter, or similar and at least one tube, pathway or similar, through which the extracted blood circulates, wherein each tube is connected to, at least, one means for collecting blood, said blood falling into the collection means by the effect of gravity and the collection means being connected to, at least, one suction means.

One object of the present invention is a device for extracting blood from the placenta and umbilical cord which allows the aforesaid extraction to be carried out by piercing the umbilical cord without having to make any puncture in the placenta.

In a preferred embodiment of the present invention, the blood extraction means include, at least one needle, catheter or similar and a tube, pathway or similar as backup. In this way it is possible to have a backup system directly attached to the extraction device, for use in case of need, without having to replace any additional element, which could delay or complicate the extraction process.

In a preferred embodiment of the present invention, at least one of the tubes has a closing mechanism, preferably through clamp-type grips, or alternatively valve or by-pass systems, manually or automatically actuated. This provides means for controlling and retaining the extracted blood.

In a preferred embodiment of the present invention, the collection means contain anticoagulants inside. This allows the extracted blood to be preserved in optimum conditions.

In a preferred embodiment of the present invention, the suction means can be operated manually or automatically. This provides an adaptable device for varying functionalities, according to the requirements of each extraction process.

Other characteristics and benefits of the present invention will be inferred from the following description, as well as the illustrative embodiment of the accompanying drawing.

### BRIEF DESCRIPTION OF THE DRAWINGS

FIG. 1 is a schematic representation of an embodiment of the device described by the present invention.

### DETAILED DESCRIPTION OF THE PREFERRED EMBODIMENTS

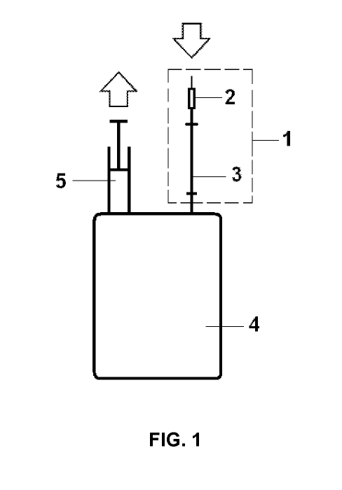

In accordance with the invention, and following the embodiment represented in FIG. 1, the device for extracting and collecting blood consists of, at least, one blood extraction means (1) which comprises, preferably, at least, one needle, catheter or similar (2) designed to pierce the umbilical vein and/or venous system of the placenta and, at least, one tube, pathway or similar (3) through which the extracted blood circulates. In different embodiments of the present invention, it is possible to have more than one tube and needle as a backup system, in case of need. In another preferred embodiment, the tube (3) has a closing mechanism, preferably, through clamp-type grips. Each tube (3) is connected to, at least, one blood collection means (4), which preferably consists of a collection sack incorporating anticoagulants inside in order to preserve the blood. Each collection means (4) is connected, in turn, to at least one suction means (5), which preferably consists of a plunger system, manually or automatically operated.

The procedure for extracting blood carried out by means of the present invention therefore comprises the following stages:

a) Piercing the umbilical vein and/or venous system of the placenta with the needle (2). Optionally, the umbilical vein can be pierced to extract the blood contained in the placenta, without having to puncture the latter.

b) Keeping the collection means (4) at a height below that of the mother and in constant motion, favouring, respectively, the falling of blood through the effect of gravity and keeping the blood in optimum conditions.

c) Using the suction means (5) to facilitate the blood's extraction and fall due to gravity into the collection means (4), in a controlled manner, applying a sufficiently gentle negative pressure so as to prevent expulsion of the placenta, said pressure being evaluated by the doctor, according to the inherent characteristics of each patient, as well as the circumstances of the delivery.

By means of the described process it is possible therefore, to carry residual blood existing in the placenta and umbilical cord, which is not accessible using a method based exclusively on the fall by gravity. This allows the volume of blood collected to be increased significantly, said increase being estimated as being within the range of 80 to 100 ml, corresponding to an increase of between 89% and 111% against the volume collected by the usual methods based on fall due to gravity.

Additionally, the existence of suction means present in the device of the invention, which can be operated in a gentle and controlled manner and designed as a support to the method for extracting and collecting blood by gravity, is capable of providing the guarantee that the placenta is not expelled prematurely, thereby preventing the risk to the mother's health associated to the methods of extraction through suction existing in the state of the technique.

All described embodiments for the present invention must not be considered as limiting against other variations in its design or materials used in its manufacture, on condition that such variations do not alter the essence of the invention in addition to the object thereof.

1. A device for extracting blood from a placenta or umbilical cord, the device comprising: at least one blood extraction means (1) having at least, one needle or catheter (2) suitable for piercing the umbilical vein or the venous system of the placenta and at least one tube or pathway (3) through which the extracted blood circulates; at least one blood collection means (4) connected to the at least one tube or pathway for collecting the extracted blood, said blood collection means being disposed so that said blood drops into the collection means (4) through an effect of gravity; and at least one suction means connected to said collection means (4) the at least one suction means (5) comprising a plunger system.

2. (canceled)

3. The device for extracting blood according to claim 1, characterized in that the blood extraction means (1) further includes at least one backup needle (2) and tube (3).

4. The device for extracting blood according to claim 3, characterized in that at least one of the tubes (3) has at least one a closing mechanism.

5. The device for extracting blood according to claim 1, characterized in that the collection means (4) contain, inside, anticoagulants.

6. The device for extracting blood according to claim 1, characterized in that the suction means (5) can be operated manually or automatically.

7. The device for extracting blood according to claim 4, wherein the closing mechanism comprises a clamp-type grips.
